(19) United States
(12) Patent Application Publication
(10) Pub. No.: US 2026/0354664 A1
(43) Pub. Date: Nov. 12, 2026

(54) MEDICAL IMAGING DEVICES, SYSTEMS, AND RELATED METHODS OF MANUFACTURING

(71) Applicant: Boston Scientific Scimed, Inc., Maple Grove, MN (US)

(72) Inventors: Eric Noble LENT, Roberts, WI (US); John Joseph SUBASIC, Sutton, MA (US)

(73) Assignee: Boston Scientific Scimed, Inc., Maple Grove, MN (US)

(21) Appl. No.: 19/668,337

(22) Filed: May 5, 2026

Related U.S. Application Data
(60) Provisional application No. 63/802,135, filed on May 8, 2025.

(51) Int. Cl. A61B 1/00; A61B 1/018; A61B 1/05; A61B 1/06; A61M 25/00

(52) U.S. Cl. CPC A61B 1/00096; A61B 1/018; A61B 1/051; A61B 1/0676; A61M 25/0097

(57) ABSTRACT

Medical devices are described, including a medical device that includes a hub, a shaft extending distally from the hub to a distal end, and a cap coupled to the distal end of the shaft. The shaft may include a working channel extending through the shaft and terminating at a distal opening proximal to a distal-most face of the shaft. The cap may include a first portion extending distally from the distal-most face of the shaft and a second portion received within the distal end of the shaft. The first portion may include an imaging assembly angled relative to a longitudinal axis of the cap. The imaging assembly may include an imaging device and a light source mounted on a printed circuit board (PCB).

### CROSS-REFERENCE TO RELATED APPLICATIONS

This application claims the benefit of priority to U.S. Provisional Application No. 63/802,135, filed on May 8, 2025, which is incorporated by reference herein in its entirety.

### TECHNICAL FIELD

This disclosure relates generally to medical devices and systems with imaging components, and methods of manufacturing such medical devices. In particular, aspects of this disclosure relate to medical devices with imaging components useful for visualization within a subject's body during a medical procedure.

### BACKGROUND

Medical procedures often use imaging devices to view or image internal body anatomy of a subject. Medical procedures may use imaging devices such as endoscopes, cystoscopes, or other medical devices that have imagers or light sources for visualization. For example, urological procedures, such as cystoscopy, may use a rigid cystoscope including rod lenses for visualization. Current imaging devices may have costly or complex manufacturing processes.

### SUMMARY

This disclosure includes a medical device that includes a hub, a shaft extending distally from the hub to a distal end, and a cap coupled to the distal end of the shaft. The shaft may include a working channel extending through the shaft and terminating at a distal opening proximal to a distal-most face of the shaft. The cap may include a first portion extending distally from the distal-most face of the shaft and a second portion received within the distal end of the shaft. The first portion may include an imaging assembly angled relative to a longitudinal axis of the cap. The imaging assembly may include an imaging device and a light source mounted on a printed circuit board (PCB).

According to some aspects, the PCB may include a distal-facing surface and a proximal-facing surface, and each of the imaging device and the light source may be mounted on the distal-facing surface of the PCB. In some examples, the imaging assembly may be disposed on an angled surface of the first portion. In some examples, the angle of the imaging assembly relative the longitudinal axis of the cap may be from about 10 degrees to about 50 degrees. In some examples, the first portion of the cap may include a distal-most surface angled relative to the longitudinal axis of the cap. The imaging device may extend to the distal-most surface, and each of the PCB and the light source may be proximal to the distal-most surface. In some examples, the first portion of the cap may include an encapsulate material completely covering each of a distal-facing surface of the PCB and the light source. The encapsulate material may only partially cover the imaging device. In some examples, the distal-most surface of the cap may be a planar surface including a distally facing surface of a lens of the imaging device, and portions of the encapsulate material. In some examples, the encapsulate material may include an optically clear epoxy. In some examples, the first portion may include a body containing the imaging assembly. The second portion may include a cylindrical body extending proximally from the body of the first portion. In some examples, a cross-sectional shape of the body of the first portion may be semi-annular or semi-circular. In some examples, the working channel may be a first channel, and the shaft may further include a second channel terminating at a distal opening at the distal-most face of the shaft. The second portion of the shaft may be received within the second channel. In some examples, the cap may include a channel extending proximally from the PCB. The channel of the cap may be in communication with the second channel of the shaft. One or more wires may extend proximally from the PCB, through the channel of the cap, through the second channel of the shaft, to the hub. In some examples, the PCB may be a first PCB, and the hub may include a second PCB. The one or more wires may extend proximally from the first PCB to the second PCB. In some examples, the distal opening of the working channel may be angled relative to the distal-most face of the shaft.

This disclosure also includes, for example, a cap for a medical device that includes a first portion, a second portion, and an optically clear material. The first portion may include a body and an imaging assembly. The body may include an inner surface recessed from a distal-most end of the body. The surface may be angled relative to a longitudinal axis of the cap. The imaging assembly may be disposed on the inner surface of the body. The imaging assembly may include an imaging device and a light source mounted on a distal-facing surface of a printed circuit board (PCB). A proximal-facing surface of the PCB may contact the inner surface of the body. The second portion may include a cylindrical body extending proximally from the body of the first portion. The cylindrical body may be configured to be received within a distal end of a shaft of a medical device. The optically clear material may be within the body. The optically clear material may cover portions of the imaging assembly. In some examples, a distal-most surface of the cap may be a planar surface. The planar surface may include portions of the optically clear material and a distally facing surface of a lens of the imaging device that may be free of the optically clear material.

This disclosure also includes, for example, a medical device that includes a shaft having a distal portion, a cap coupled to the distal portion of the shaft, and an encapsulate material. The cap may include a first portion extending distally from a distal-most end of the shaft, and a second portion including a cylindrical body received within the distal portion of the shaft. The first portion may include a body and an imaging assembly. The body may include an inner wall recessed from a distal-most end of the body. The inner wall may include a distally facing surface that is angled relative to a longitudinal axis of the shaft. The imaging assembly may be disposed on the distally facing surface of the inner wall. The imaging assembly may include an imaging device and a light source mounted on a printed circuit board (PCB). The encapsulate material may cover only portions of the imaging assembly. In some examples, the encapsulate material may extend through an opening in the inner wall of the body of the first portion. In some examples, the encapsulate material may completely surround the cylindrical body of the second portion. In some examples, the encapsulate material may completely fill an interior of the cylindrical body.

### BRIEF DESCRIPTION OF FIGURES

The accompanying drawings, which are incorporated in and constitute a part of this application, illustrate exemplary aspects of this disclosure and together with the description, serve to explain the principles of this disclosure.

FIG. 1A illustrates an exemplary medical device, in accordance with some aspects of this disclosure.

FIG. 1B illustrates a distal portion of the medical device of FIG. 1A, in accordance with some aspects of this disclosure.

FIG. 1C illustrates a cross-sectional view of the distal portion of the medical device of FIG. 1A, in accordance with some aspects of this disclosure.

FIG. 1D illustrates an exemplary electronic assembly and an exemplary cap of the medical device of FIG. 1A, in accordance with some aspects of this disclosure.

FIG. 1E illustrates a proximal portion of the medical device of FIG. 1A, in accordance with some aspects of this disclosure.

FIG. 2 illustrates a cross-sectional view of distal portion of an exemplary medical device, in accordance with some aspects of this disclosure.

FIG. 3 illustrates a partial cross-sectional view of a distal portion of another exemplary medical device, in accordance with some aspects of this disclosure.

FIG. 4 illustrates a distal portion of a further exemplary medical device, in accordance with some aspects of this disclosure.

FIG. 5 illustrates a partial cross-sectional view of a distal portion of another exemplary medical device, in accordance with some aspects of this disclosure.

FIG. 6 illustrates a distal portion of yet another exemplary medical device, in accordance with some aspects of this disclosure.

FIGS. 7A-7B illustrate an exemplary medical system, in accordance with some aspects of this disclosure.

### DETAILED DESCRIPTION

Particular aspects of the disclosure are described in greater detail below. The terms and definitions provided herein control, if in conflict with terms and/or definitions incorporated by reference. Wherever possible, the same or similar reference numbers will be used through the drawings to refer to the same or like parts. Where feasible, reference numbers ending in the same tens and ones digits refer to corresponding components.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “or” is used disjunctively, such that “at least one of A or B” includes, (A), (B), (A and A), (A and B), etc. The term “or” also includes “and/or.” Relative terms such as “about,” “substantially,” and “approximately,” etc., are used to indicate a possible variation of ±10% of the stated numeric value or range.

This disclosure includes medical devices having imaging capabilities useful for viewing or imaging internal body anatomy of a subject (e.g., a patient). According to some aspects of this disclosure, the medical devices herein may include a shaft and an imaging assembly. The imaging assembly may include an imaging device, one or more light sources, and a printed circuit board (PCB). The imaging assembly may be disposed within a distal end of the shaft or may be coupled to the distal end of the shaft via a cap. The medical devices herein may provide a desired field of view for a particular medical procedure or application. For example, the imaging device may be angled relative to a longitudinal axis of the shaft or may be parallel to the longitudinal axis of the shaft.

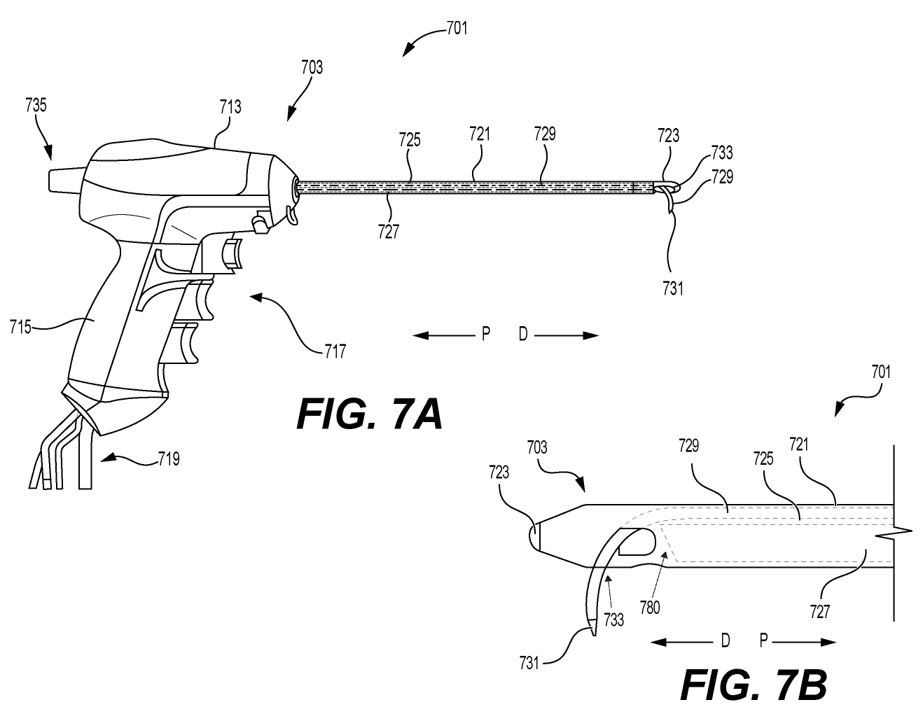

According to some aspects of this disclosure, the medical devices herein may be compatible with other medical devices. For example, the medical devices herein may be inserted into a shaft of another medical device. For example, the medical devices herein may be used with medical devices for treating benign prostatic hyperplasia, such as Rezum^{TM}. The medical devices herein may be used in conjunction with, e.g., the system illustrated in FIGS. 7A and 7B.

Methods of manufacturing the medical devices herein are also described. The methods of manufacturing and materials described may help reduce manufacturing costs or complexity, while maintaining or enhancing imaging capabilities of the medical devices herein.

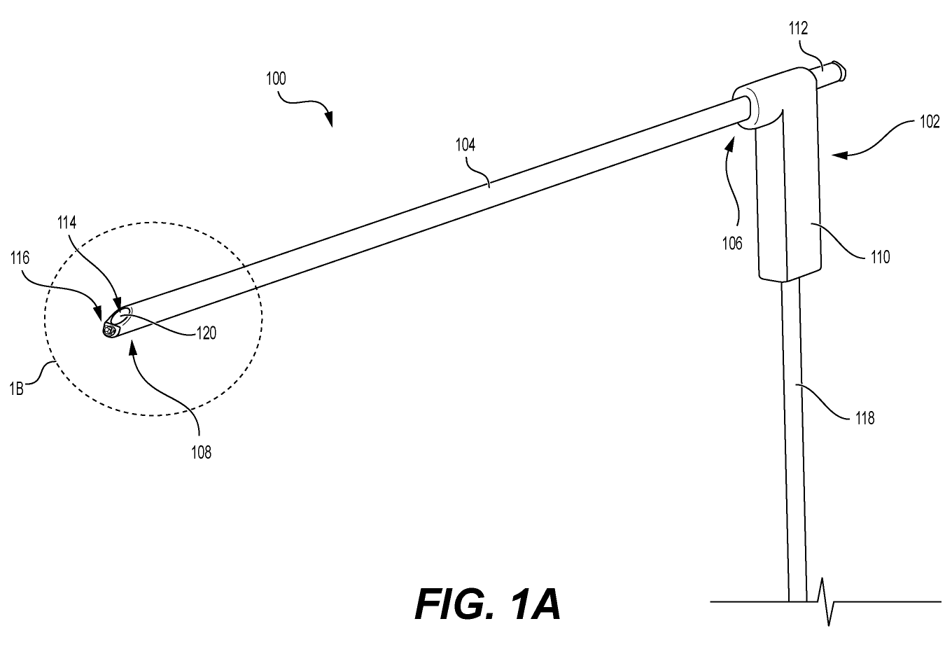

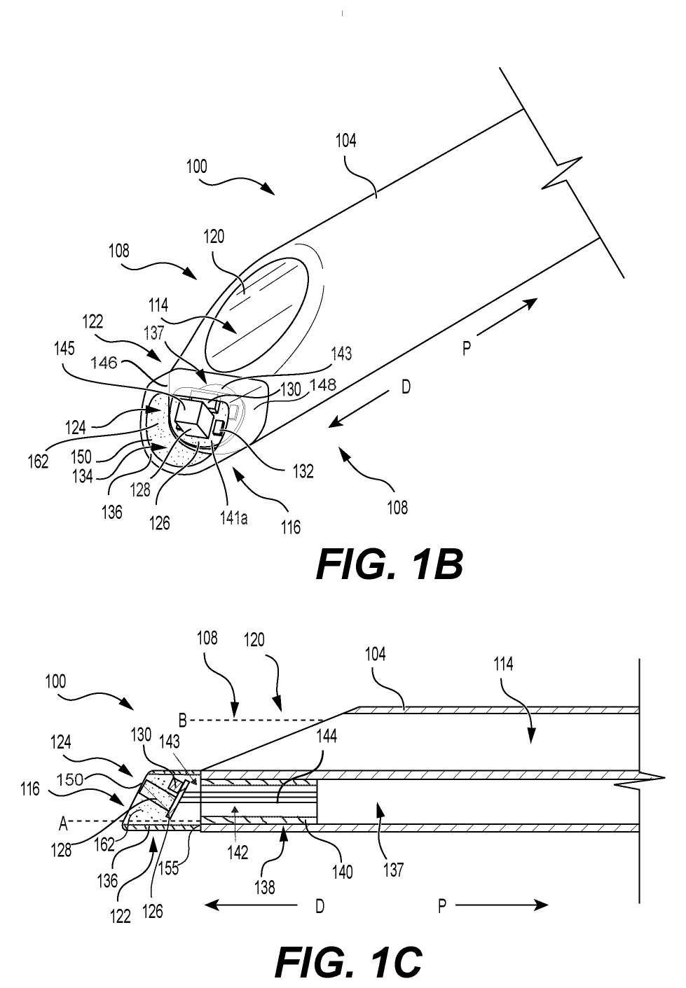

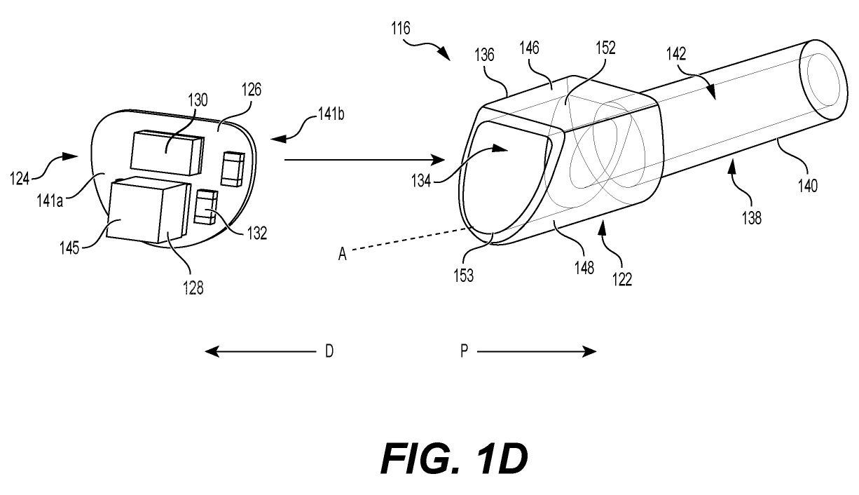

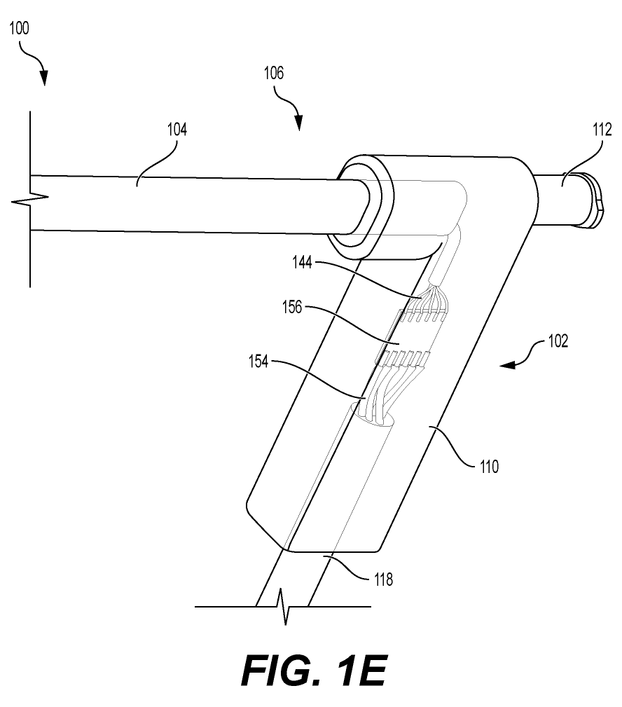

An exemplary medical device 100 will now be described with reference to FIGS. 1A-1E. FIG. 1A illustrates a perspective view of medical device 100. FIG. 1B illustrates a perspective view of a distal portion of medical device 100. FIG. 1C illustrates a cross-sectional view of the distal portion of medical device 100. FIG. 1D illustrates perspective views of a cap 116 and an imaging assembly 124 (an electronic assembly) of medical device 100 unassembled. FIG. 1E illustrates a perspective view of a proximal portion of medical device 100.

Referring to FIG. 1A, medical device 100 may include a hub 102, a shaft 104, and a cap 116. Shaft 104 may extend from a proximal end 106 that is coupled to hub 102 to a distal end 108. Cap 116 may be coupled to distal end 108 of shaft 104 and may house imaging assembly 124. Shaft 104 may include a working channel 114 (e.g., a first channel) extending from proximal end 106 to distal end 108. Working channel 114 may terminate at a distal opening 120 at distal end 108 of shaft 104.

As more clearly shown in FIGS. 1B and 1C, shaft 104 may include a channel 137 (e.g., a second channel) extending from proximal end 106 to distal end 108 of shaft 104. Channel 137 may terminate at a distal opening 143 at a distal-most face 155 of shaft 104. Distal-most face 155 of shaft 104, including distal opening 143 of channel 137, may extend substantially perpendicularly to a longitudinal axis B of shaft 104. Distal opening 120 of working channel 114 (and surfaces of shaft 104 immediately surrounding distal opening 120) may be angled non-perpendicularly relative to longitudinal axis B of shaft 104 or relative to distal-most face 155 of shaft 104. For example, distal opening 120 of working channel 114 may be proximal to distal-most face 155. Channels 114 and 137 of shaft 104 may be separate channels extending longitudinally through shaft 104, e.g., channels 114 and 137 may not be in fluid communication. Portions of cap 116 and electrical wires or cables may extend through channel 137 of shaft 104, as will be further discussed below.

Referring to FIG. 1A, hub 102 may include a housing 110 and a port 112 that is in fluid communication with working channel 114. A suitable medical instrument or tool may be inserted through port 112 and moved distally through shaft 104 via working channel 114. In some examples, port 112 may be coupled to a source of fluid to deliver a fluid (e.g., air or water) through working channel 114 or may be coupled to a source of suction to apply suction through working channel 114. In some examples, hub 102 may function as a handle for manually grasping or actuating (e.g., rotating) shaft 104 during a medical procedure. In other examples, hub 102 may assist with connecting medical device 100 to another medical device for use during a medical procedure, as will be further discussed below with reference to FIGS. 7A and 7B.

Hub 102 may be coupled to a cable 118 (e.g., an umbilicus). Cable 118 may extend from hub 102 and may include or carry wires configured to provide, e.g., power or signals to or from hub 102. For example, cable 118 may connect hub 102 to capital equipment, such as electronic controller(s), one or more displays, or a power supply. Cable 118 may be a low-cost cable, e.g., a USB cable or an HDMI cable.

Referring to FIGS. 1B-1D, cap 116 may include a first portion 122 (e.g., a distal portion) and a second portion 138 (e.g., a proximal portion). Second portion 138 may be narrower than first portion 122 (have a smaller width in a direction perpendicular to longitudinal axis B). First portion 122 of cap 116 may extend distally from distal-most face 155 of shaft 104, and second portion 138 may be disposed within channel 137 of shaft 104. Accordingly, first portion 122 of cap 116 may not extend around an outer circumference of shaft 104 and may be disposed entirely distally of shaft 104. First portion 122 of cap 116 may cover distal-most face 155, including distal opening 143 of channel 137, but may not cover distal opening 120 of working channel 114. For example, cap 116 may not be in communication with working channel 114. First portion 122 of cap 116 may be disposed only covering a portion of shaft 104 having distal-most face 155 and not other portions of shaft 104 defining working channel 114.

First portion 122 of cap 116 may include a distal-most surface 150 (e.g., a distal-most surface of cap 116). As more clearly shown in FIG. 1C, distal-most surface 150 may be angled relative to longitudinal axis B of shaft 104 or relative to a longitudinal axis A of cap 116. In some examples, an angle of distal-most surface 150 relative to longitudinal axis A or B may be from about 0 degrees to about 90 degrees, e.g., about 5 degrees to about 85 degrees, or about 10 degrees to about 50 degrees, or about 30 degrees. As will be further discussed below, distal-most surface 150 may be a planar surface that includes a distally facing surface of a lens 145 of imaging device 128 and portions of a material 162.

First portion 122 of cap 116 may include a body 136 containing imaging assembly 124. Imaging assembly 124 may include imaging or lighting components mounted on a printed circuit board (PCB) 126. Imaging assembly 124 may include an imaging device 128 (e.g., a camera), one or more light sources 130 (e.g., light-emitting diode(s)), or one or more other electronic components 132 (e.g., a capacitor, a resistor, or other electronic component) mounted on a distal-facing surface 141a of PCB 126. Imaging device 128 may extend to distal-most surface 150, while PCB 126, light source(s) 130, and other electronic component(s) 132 may be proximal to distal-most surface 150. Body 136 of first portion 122 may be optically clear to allow light in the visible range to pass through, which may help maximize light brightness and minimize shadows during a medical procedure. In some examples, both body 136 of first portion 122 and a cylindrical body 140 of second portion 138 may be optically clear. In other examples, in addition to body 136 or cylindrical body 140 being optically clear, shaft 104 may also be optically clear.

As more clearly shown in FIG. 1C, imaging assembly 124 may be angled relative to longitudinal axis B of shaft 104 or relative to longitudinal axis A of cap 116, e.g., to provide a desired viewing angle for imaging device 128. For example, imaging assembly 124 may be aimed toward working channel 114. When inserted into, e.g., a medical system 701, as discussed below, imaging assembly 124 may be aimed toward a needle 729 so that imaging device 128 may visualize needle 729. Imaging assembly 124 may be disposed on an angled, inner surface 152 of body 136 (shown in FIG. 1D) that is recessed from a distal-most end of body 136. An angle of imaging assembly 124 relative to longitudinal axis A or B may correspond to an angle of inner surface 152 relative to longitudinal axis A or B. For example, an angle of inner surface 152 relative to longitudinal axis A or B may be from about 0 degrees to about 90 degrees, e.g., about 5 degrees to about 85 degrees, or about 10 degrees to about 50 degrees, or about 30 degrees.

As mentioned above, first portion 122 may extend distally from distal-most face 155 of shaft 104. Body 136 of first portion 122 may have a size and a shape that corresponds to a size and a shape of distal-most face 155. Distal-most face 155 may have a semi-annular or semi-circular shape, and a cross-sectional shape of body 136 of first portion 122 may be semi-annular or semi-circular. For example, body 136 of first portion 122 may include a planar surface 146 and a U-shaped surface 148 (e.g., a semi-tubular surface). Planar surface 146 may extend substantially parallel to longitudinal axis B of shaft 104, and U-shaped surface 148 may extend between lateral ends of planar surface 146. U-shaped surface 148 may be aligned with an outer surface of shaft 104. For example, U-shaped surface 148 may not extend beyond an outer surface of shaft 104. U-shaped surface 148 may provide cap 116 with a smooth, rounded exterior that may reduce the risk of damage to surrounding tissue when shaft 104 is inserted into a body lumen of a subject. U-shaped surface 148 may also be complementary to a shape of a bore through which medical device 100 is inserted (e.g., a bore of system 701).

Cap 116 may include material 162 (e.g., an encapsulate material, a potting material, an adhesive, an epoxy, etc.) within a cavity 134 (discussed in further detail below) of body 136 of first portion 122. Material 162 may comprise an optically clear material, e.g., an optically clear epoxy. Portions of body 136 distal to inner surface 152 may be filled with material 162. For example, material 162 may completely cover portions of distal-facing surface 141a of PCB 126 that do not include electronic components, light source(s) 130, and other electronic component(s) 132. Material 162 may partially cover or partially surround imaging device 128. For example, material 162 may not cover a distally facing surface of lens 145 of imaging device 128, but may cover or surround remaining portions of imaging device 128. Material 162 may seal and protect imaging assembly 124 from the external environment, while also securing imaging assembly 124 in place. For example, material 162 may prevent fluid ingress. Additionally, material 162 may allow uniform scattering of light through material 162 and onto a target site (e.g., a target tissue site) during a medical procedure. Uniform scattering of light may reduce or eliminate shadows, e.g., from lens 145 of imaging device 128, from being projected into a field of view of imaging device 128.

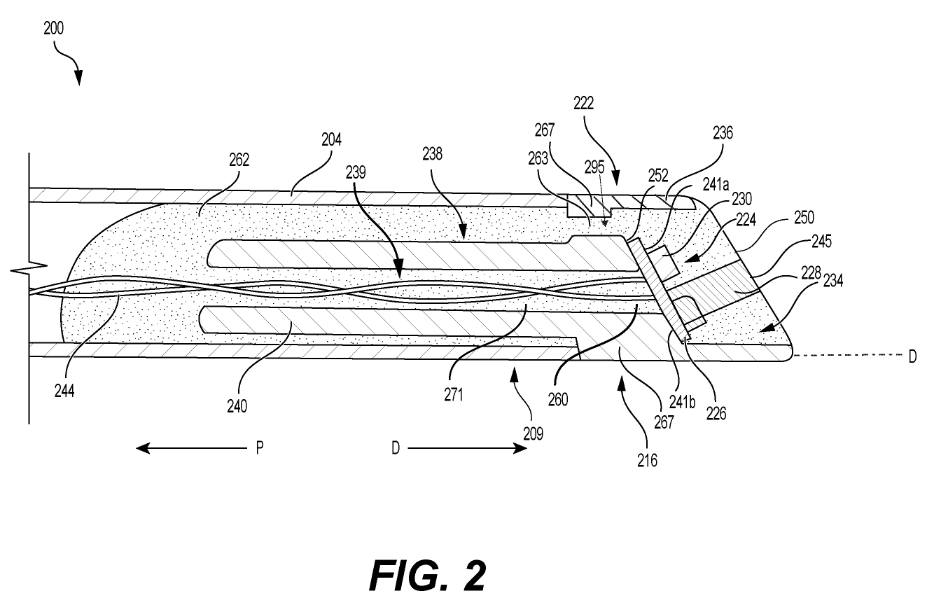

Referring to FIGS. 1C and 1D, second portion 138 of cap 116 may include a cylindrical body 140 extending proximally from body 136 of first portion 122. Cylindrical body 140 of second portion 138 may be fixed within channel 137 of shaft 104, e.g., fixed within a distal portion of channel 137. For example, cylindrical body 140 may be tightly or snuggly fitted within channel 137. In some examples, cylindrical body 140 may be fitted into channel 137 solely by friction. In other examples, cylindrical body 140 and channel 137 may include corresponding locking features (e.g., a barb, a ledge) to secure cylindrical body 140 within channel 137. In yet another example, cylindrical body 140 and shaft 104 may be ultrasonically welded or reflowed together during manufacturing to secure cylindrical body 140 to distal end 108 of shaft 104. Alternatively, cylindrical body 140 may be secured within channel 137 by adhesive (e.g., a glue, material 162, etc.). In at least one example in which material 162 is used to secure cylindrical body 140 within channel 137, cap 116 may include openings or channels similar to openings or channels of a cap 216 (which will be discussed below with reference to FIG. 2) to facilitate application or flow of material 162 around cylindrical body 140 and within channels 137, 142.

Cap 116 may include a channel 142 that extends through portions of body 136 and cylindrical body 140. For example, channel 142 may extend proximally from PCB 126, through body 136, and through cylindrical body 140. Channel 142 may be in communication with channel 137 of shaft 104 and may accommodate one or more wires 144 extending proximally from PCB 126.

Wire(s) 144 may extend proximally from PCB 126, through channels 142, 137, to hub 102. Wire(s) 144 may connect PCB 126 and the electronic components (e.g., imaging device 128, light source(s) 130, other electronic component(s) 132) on PCB 126 to hub 102 or to a connector at an end of cable 118.

FIG. 1E illustrates a transparent view of hub 102. As shown, wire(s) 144 may extend proximally from PCB 126 (e.g., a first PCB, a distal PCB) to a printed circuit board (PCB) 156 (e.g., a second PCB, a proximal PCB) within housing 110 of hub 102. PCB 156 may be also coupled to one or more wires 154 of cable 118. Wire(s) 144, 154 may be soldered or otherwise coupled to PCB 156. Coupling of wire(s) 144, 154 to PCB 156 may electronically couple PCB 126 and the electronic components on PCB 126 to capital equipment (e.g., electronic controller(s), a display, a power source). For example, signals may be transmitted between PCB 126 and capital equipment via PCB 156 for controlling imaging device 128 or light source(s) 130, or for displaying an image on a display. PCB 156 may include a decoupling capacitor or other electronic components configured to improve imaging performance of medical device 100.

Hub 102 may be formed via an injection molding process. For example, hub 102 may be formed via an injection molding process around proximal end 106 of shaft 104, and around wire(s) 144, 154 and PCB 156. The injection molding process may provide strain relief and may help facilitate securing the electrical connections between wire(s) 144, 154 and PCB 156 within hub 102. Additionally, the injection molding process may help facilitate sealing the electrical connections between wire(s) 144, 154 and PCB 156 from the external environment, e.g., to prevent fluid ingress.

An exemplary method of assembling imaging assembly 124, cap 116, and shaft 104 will now be described with reference to FIG. 1D. Imaging assembly 124 and cap 116 are shown unassembled in FIG. 1D. In an unassembled configuration, body 136 of first portion 122 may define cavity 134 and a distal opening 153 that is in communication with cavity 134. Distal opening 153 may be at a distal end of body 136 (e.g., a distal end of cap 116). The distal end of body 136 may be angled relative to longitudinal axis A of cap 116. For example, an angle of the distal end of body 136 relative to longitudinal axis A of cap 116 may be from about 0 degrees to about 90 degrees e.g., about 5 degrees to about 85 degrees, or about 10 degrees to about 50 degrees, or about 30 degrees. Cavity 134 may be sized and shaped to receive imaging assembly 124. For example, PCB 126 and cavity 134 may each have a semi-annular or semi-circular shape.

During assembly, wire(s) 144 may be soldered or otherwise attached to proximal-facing surface 141b of PCB 126. Optionally, strain relief may be added to strengthen the connection between wire(s) 144 and PCB 126. Wire(s) 144 may then be pulled through channel 142 of cap 116 and channel 137 of shaft 104.

PCB 126 may then be aligned with distal opening 153 of body 136, such that proximal-facing surface 141b of PCB 126 faces distal opening 153 of body 136. PCB 126 may then be inserted into distal opening 153 and into cavity 134 of body 136. PCB 126 may be pushed into cavity 134 of body 136 until proximal-facing surface 141b of PCB 126 contacts or rests on angled, inner surface 152 of body 136. Once proximal-facing surface 141b of PCB 126 contacts or rests on inner surface 152, imaging assembly 124 may now also be angled, with the distally facing surface of lens 145 of imaging device 128 being aligned with distal opening 153 of body 136.

A fixture may then be placed over the distal end of body 136. For example, the fixture may be placed over distal opening 153 of body 136. The fixture may include means to allow material 162 to be injected into cavity 134 of body 136. Once the fixture is secured in place over distal opening 153 of body 136, material 162, in a liquid, fluid, or free-flowing form, may be injected into cavity 134 of body 136. Material 162, in the liquid, fluid, or free-flowing form, may be injected until cavity 134 of body 136 is adequately filled with material 162. Material 162 may completely cover portions of distal-facing surface 141a of PCB 126 that do not include electronic components, light source(s) 130, and other electronic component(s) 132. Material 162 may partially cover imaging device 128. Due to the distally facing surface of lens 145 being aligned with distal opening 153 of body 136, material 162 may not be applied to the distally facing surface of lens 145. Quality of images (e.g., clarity of images) captured by imaging device 128 may be improved by keeping material (e.g., material 162) out of the field of view of imaging device 128.

Once cavity 134 of body 136 is adequately filled with material 162, material 162 may be cured, e.g., via UV curing or other curing process, to harden material 162. After material 162 is cured, the fixture may be removed from the distal end of body 136, thereby exposing distal-most surface 150 that is angled relative to longitudinal axis A of cap 116. The distally facing surface of lens 145 that is free of material 162 may be flush with portions of material 162 that now occupy distal opening 153 of body 136, thus forming distal-most surface 150.

A glue or other suitable adhesive may then optionally be applied to cylindrical body 140 of second portion 138. Cylindrical body 140 of second portion 138 may be inserted into channel 137 of shaft 104 via distal opening 143 securing cap 116 to distal end 108 of shaft 104. Due to cylindrical body 140 being fixed to channel 137, cap 116 may be inhibited from rotating or otherwise moving relative to shaft 104. For example, cap 116 may be inhibited from dislodging from shaft 104 during a medical procedure or inadvertently changing a field of view of imaging device 128.

FIG. 2 illustrates a cross-sectional view of a distal portion of an exemplary medical device 200 that includes a shaft 204 and cap 216 coupled to a distal portion 209 of shaft 204. Shaft 204 may include any of the features of shaft 104, and cap 216 may include any of the features of cap 116, unless otherwise specified herein.

In this example, shaft 204 may not include a working channel, e.g., working channel 114. Shaft 204 may include a material 262 (e.g., an optically clear material, such as an optically clear epoxy) similar to material 162 within distal portion 209 of shaft 204, as will be further discussed below. Shaft 204 and cap 216 may be used in combination with hub 102, e.g., in place of shaft 104 and cap 116, and without port 112.

Cap 216 may include a first portion 222 (e.g., a distal portion) and a second portion 238 (e.g., a proximal portion). First portion 222 may include a body 236 extending distally from a distal-most end of shaft 204. For example, body 236 may form an extension of shaft 204, and an outer surface of body 236 may not extend radially beyond an outer surface of shaft 204. In other words, an outer radial width or diameter of body 236 may be the same as an outer radial width or diameter of shaft 204. First portion 222 of cap 216 may include a distal-most surface 250 (e.g., a distal-most surface of cap 216), which may be angled relative to a longitudinal axis D of shaft 204. For example, an angle of distal-most surface 250 relative to longitudinal axis D of shaft 204 may be from about 0 degrees to about 90 degrees, e.g., about 5 degrees to about 85 degrees, or about 10 degrees to about 50 degrees, or about 30 degrees. Distal-most surface 250 may be formed at least partially from material 262.

Body 236 may contain an imaging assembly 224 similar to imaging assembly 124 that is disposed on an angled, inner surface 252 of body 236. An angle of inner surface 252 relative to longitudinal axis D of shaft 204 may be from about 0 degrees to about 90 degrees, e.g., about 5 degrees to about 85 degrees, or about 10 degrees to about 50 degrees, or about 30 degrees. Inner surface 252 may be a distally facing surface of an inner wall 267 of body 236 that is recessed from a distal-most end of body 236 (e.g., a distal-most end of cap 216).

Imaging assembly 224 may include a printed circuit board (PCB) 226 similar to PCB 126, an imaging device 228 similar to imaging device 128, and one or more light sources 230 similar to light source(s) 130. Imaging assembly 224 may also include other electronic components on PCB 226. Due to angled, inner surface 252, imaging assembly 224 may be angled relative to longitudinal axis D of shaft 204. As explained above for medical device 100, and will also be explained below, an angle of inner surface 252 and a corresponding angle of imaging assembly 224 may be such that imaging assembly 224 may view aspects of medical system 701 (e.g., needle 729) in operation. For example, imaging assembly 224 may be at an angle of about 0 degrees to about 90 degrees, e.g., about 5 degrees to about 85 degrees, or about 10 degrees to about 50 degrees, or about 30 degrees, relative to longitudinal axis D of shaft 204. Imaging device 228 may extend to distal-most surface 250, while PCB 226 and light source(s) 230 may be proximal to distal-most surface 250.

Second portion 238 of cap 216 may include a cylindrical body 240 extending proximally from body 236 of first portion 222, and may be disposed within distal portion 209 of shaft 204. One or more wire 244 similar to wire(s) 144 may extend proximally from PCB 226, through cylindrical body 240, through shaft 204, to hub 102. As compared with second portion 138, which fits snugly within channel 137, second portion 238 may have clearance around it within a lumen of distal portion 209, to allow for material 262 to flow around second portion 238, as described below.

Second portion 238 and a proximal portion of body 236 of first portion 222 may define an inner lumen 239 extending longitudinally through second portion 238. Lumen 239 may terminate distally at inner surface 252. Wire(s) 244 may extend through lumen 239 to PCB 226. Body 236 may also define a channel 295 that extends longitudinally through body 236. A central longitudinal axis of lumen 239 may be offset from (e.g., approximately parallel to but not coaxial with) a central longitudinal axis of shaft 204. Channel 295 may be radially outside of second portion 238 (relative to a central longitudinal axis of lumen 239) and to a side of second portion 238 that has a greater distance between second portion 238 and an inner wall of shaft 204. A longitudinal axis drawn through channel 295 may not intersect PCB 226. In other words, PCB 226 and channel 295 may not overlap one another.

Material 262 may be within distal portion 209 of shaft 204, within portions of cap 216, and may surround or cover portions of cap 216. Material 262 may seal electronic components within shaft 204 and cap 216 from the external environment. For example, material 262 may seal electronic components on distal-facing and proximal-facing surfaces 241a, 241b of PCB 226, and wire(s) 244. Material 262 may also facilitate securing cap 216 to shaft 204 and may provide strain relief for wire(s) 244.

Material 262 may completely cover exposed portions (portions not having electronic components) of distal-facing surface 241a of PCB 226 and light source(s) 230. Material 262 may partially cover or partially surround imaging device 228. For example, material 262 may not cover a distally facing surface of a lens 245 of imaging device 228, but may cover or surround remaining portions of imaging device 228. Distal-most surface 250 may be a planar surface that includes the distally facing surface of lens 245 and portions of material 262. For example, the distally facing surface of lens 245 may be flush with portions of material 262.

Material 262 may be injected into shaft 204 and cap 216 from a location (e.g., an opening in an outer wall of shaft 204) that is proximal of cap 216 or through a distal open end of body 236. In one aspect, material 262 may be injected so that it flows distally into lumen 239 until it abuts proximal-facing surface 241b of PCB 226. Material 262 may also flow distally through channel 295 until it fills a cavity 234 defined by body 236 at a distal end of cap 216. Thus, material 262 may cover portions of proximal-facing surface 241b of PCB 226. For example, first portion 222 of cap 216 may include a first section 260 of material 262 that is underneath (proximal of) PCB 226 and that extends through lumen 239. First portion 222 of cap 216 may include a second section 263 of material 262 that extends through channel 295.

Cylindrical body 240 may be fixed within or otherwise secured within distal portion 209 of shaft 204 via material 262. As shown, material 262 may completely surround or cover cylindrical body 240. For example, material 262 may completely surround or cover an exterior of cylindrical body 240. Material 262 may completely surround or cover a circumference of cylindrical body 240 as well as a proximal end of cylindrical body 240.

Additionally, material 262 may completely fill lumen 239. For example, second portion 238 of cap 216 may include a third section 271 of material 262 extending through lumen 239 that is continuous with first section 260 of material 262. As mentioned above, wire(s) 244 may extend proximally from PCB 226. As shown, at least distal portions of wire(s) 244 may be surrounded by material 262.

Once cap 216 and distal portion 209 of shaft 204 are adequately filled with material 262, material 262 may be cured to harden material 262. As mentioned above, material 262 may provide strain relief for wire(s) 244, and may eliminate the need to add an extra strain relief process step for wire(s) 244 during the manufacturing process. Material 262 may also seal various components from fluids and secure various components in place.

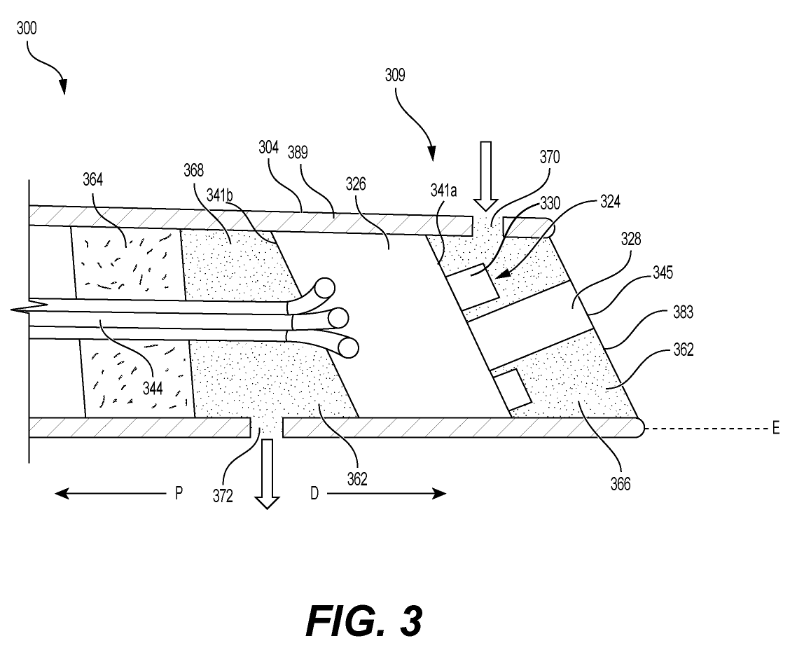

According to some aspects of this disclosure, the medical devices herein may include an imaging assembly integrated within a shaft instead of a cap. Such medical devices are illustrated in FIGS. 3-6. Unless otherwise provided below, the medical devices herein may have any of the properties of medical devices 100 or 200. Any of the above or below aspects may be combined in various ways.

FIG. 3 illustrates a partial cross-sectional view of a distal portion of an exemplary medical device 300 that includes a shaft 304 and an imaging assembly 324 within a distal portion 309 of shaft 304. Imaging assembly 324 may include a printed circuit board (PCB) 326, an imaging device 328, and one or more light sources 330. Imaging device 328 and light source(s) 330 may be mounted on a distal-facing surface 341a of PCB 326. One or more wires 344 similar to wire(s) 144, 244 may extend proximally from PCB 326 and through shaft 304. Shaft 304 may be used in combination with hub 102, e.g., in place of shaft 104 and cap 116, and without port 112.

Imaging assembly 324 may be angled relative to a longitudinal axis E of shaft 304. For example, an angle of imaging assembly 324 relative to longitudinal axis E of shaft 304 may be from about 0 degrees to about 90 degrees, e.g., about 5 degrees to about 85 degrees, or about 10 degrees to about 50 degrees, or about 30 degrees. Imaging device 328 may extend to a distal-most surface 383 of a material 362 within shaft 304, while PCB 326 and light source(s) 330 may be proximal to distal-most surface 383. Similar to imaging assembly 324, distal-most surface 383 may be angled relative to longitudinal axis E of shaft 304. In some examples, portions of an outer wall 389 of shaft 304 may extend distally of other portions, to create an angled distal-most opening for receiving imaging assembly 324.

Distal portion 309 of shaft 304 may include material 362 similar to materials 162, 262. Material 362 may completely cover distal-facing surface 341a of PCB 326 (portions that are not covered by electronic components) and proximal-facing surface 341b PCB 326. For example, distal portion 309 of shaft 304 may include a first section 366 of material 362 distal to PCB 326 and a second section 368 of material 362 proximal to PCB 326.

Material 362 may completely cover or surround light source(s) 330, and may partially cover or partially surround imaging device 328. For example, material 362 may not cover a distally facing surface of a lens 345 of imaging device 328, but may cover or surround remaining portions of imaging device 328. Distal-most surface 383 may be a planar surface that includes the distally facing surface of lens 345 and portions of material 362. For example, the distally facing surface of lens 345 may be flush with portions of material 362. Material 362 may also cover or surround portions of wire(s) 344. For example, portions of wire(s) 344 may extend through second section 368 of material 362. As shown, first and second portions 370, 372 of outer wall 389 of shaft 304 may include material 362. First portion 370 may be distal to PCB 326 and second portion 372 may be proximal to PCB 326. First and second portions 370, 372 may be on opposite sides of outer wall 389.

Distal portion 309 of shaft 304 may include a material 364 (e.g., a second material) different from material 362 (e.g., a first material). Material 364 may be disposed adjacent to and proximal to second section 368 of material 362. Material 364 may include a soft material, e.g., a foam wafer. Portions of wire(s) 344 may extend through material 364. Material 364 may assist with securing wire(s) 344 within shaft 304, and may function as a backstop during manufacturing of medical device 300.

Shaft 304 may include cavities, openings, lumens, or channels for introducing material 362. Outer wall 389 may include openings 370, 372 that material 362, in a liquid, fluid, or other free-flowing form, may flow in and out of. For example, material 362, in the liquid, fluid, or other free-flowing form, may be injected into opening 370 (as indicated by an arrow) and may flow into shaft 304 and flow out of opening 372 (as indicated by an arrow). Opening 370 may be distal to PCB 326, and opening 372 may be proximal to PCB 326. PCB 326 may have a semi-annular shape, a semi-circular shape, a C-shape, or other suitable shape that may form gaps or holes between PCB 326 and an inner surface of outer wall 389. The gaps or holes between PCB 326 and the inner surface of outer wall 389 may allow for material 362 to flow into opening 370, around PCB 326, and out of opening 372 to fill areas within shaft 304 proximal and distal to PCB 326. Additionally, or alternatively, PCB 326 may include one or more openings that allow material 362 to flow into and out of.

As mentioned above, material 364 may function as a backstop that facilitates flow of material 362 out of opening 372 (as indicated by an arrow). In alternatives, material 362 may be injected into opening 372 and flow out of opening 370, material 362 may be injected into both openings 370 and 372, or shaft 304 may include only one of openings 370, 372. Once distal portion 309 of shaft 304 is adequately filled with material 362, material 362 may be cured to harden material 362.

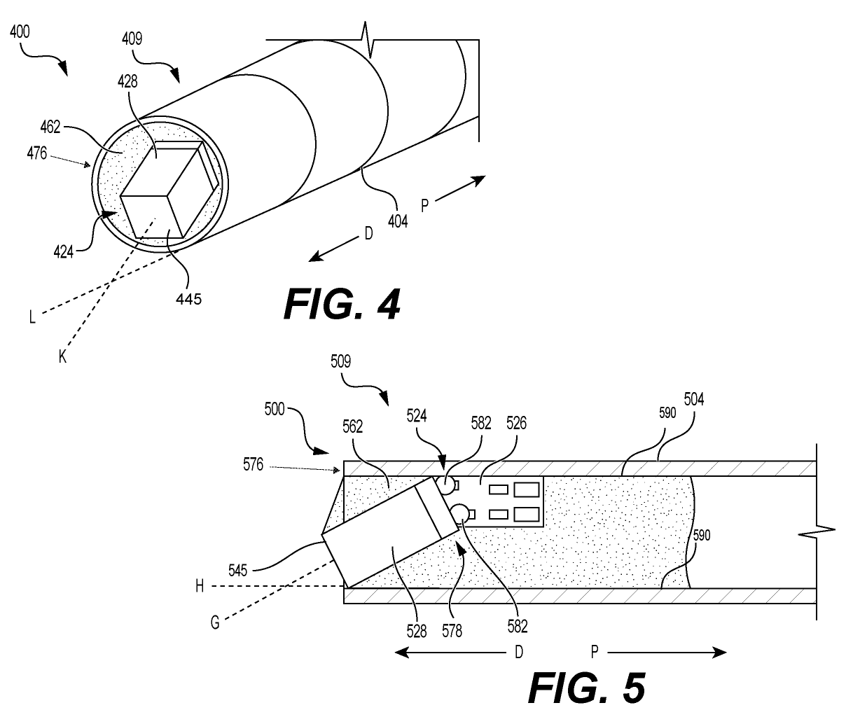

FIG. 4 illustrates a perspective view of a distal portion of an exemplary medical device 400 that includes a shaft 404 and an imaging assembly 424 within a distal portion 409 of shaft 404. Imaging assembly 424 may include an imaging device 428 that is angled relative to a longitudinal axis L of shaft 404. For example, an angle formed between a longitudinal axis K of imaging device 428 and longitudinal axis L of shaft 404 may be from about 0 degrees to about 90 degrees, e.g., about 5 degrees to about 85 degrees, or about 10 degrees to about 50 degrees, or about 30 degrees. Shaft 404 may be used in combination with hub 102, e.g., in place of shaft 104 and cap 116, and without port 112.

Distal portion 409 of shaft 404 may be filled with a material 462, similar to materials 162, 262, 362. Material 462 may surround or cover portions of imaging device 428, except for a distally facing surface of a lens 445 of imaging device 428 and a proximal side of imaging device 428. A proximal side of imaging device 428 may be coupled to, e.g., a printed circuit board, such as a two-layered printed circuit board with an offset on one side. In some examples, due to imaging device 428 being angled relative to longitudinal axis L of shaft 204, portions of imaging device 428 may protrude from a distal-most end 476 (see, e.g., the example of FIG. 5) of shaft 404. Material 462 may provide strain relief for one or more wires or cables extending proximally from imaging device 428 and through shaft 404, while also sealing electronic components within shaft 404 from the external environment.

Additionally, or optionally, imaging assembly 424 may include one or more light sources (e.g., light emitting diode(s)). For example, light source(s) may be disposed on either side of imaging device 428 to assist with visualization. Additionally, shaft 404 or imaging assembly 424 may include features, e.g., supports or mounts, that may assist with angling imaging device 428 within distal portion 409 of shaft 404.

FIG. 5 illustrates a partial cross-sectional view of a distal portion of another exemplary medical device 500 that includes a shaft 504 and an imaging assembly 524 disposed within a distal portion 509 of shaft 504. Imaging assembly 524 may include an imaging device 528 and a printed circuit board (PCB) 526. Shaft 504 may be used in combination with hub 102, e.g., in place of shaft 104 and cap 116, and without port 112.

As shown, PCB 526 may be oriented within shaft 504 such that a lateral side edge of PCB 526 may be disposed on or adjacent to an inner surface 590 of shaft 504. Imaging device 528 may be mounted to one or more edges of PCB 526. For example, a proximal end 578 of imaging device 528 may be mounted to a distal edge of PCB 526. In aspects, PCB 526 may have a non-rectangular (e.g., trapezoidal) shape, such that a distal edge of PCB 526 is at a non-perpendicular angle with respect to adjacent edges of PCB 526. For example, a distal edge of PCB 526 may have an internal angle of approximately 30 degrees with respect to one adjacent edge (e.g. an edge disposed adjacent to inner surface 590) and an internal angle of approximately 60 degrees with another adjacent edge (an edge opposite the edge disposed adjacent to inner surface 590).

Proximal end 578 of imaging device 528 may include solder balls 582 that may couple (e.g., conductively couple) imaging device 528 to PCB 526. Imaging device 528 may be angled relative to PCB 526. For example, in a distal direction, imaging device 528 may be angled towards an opposite side of inner surface 590. For example, a distal edge of imaging device 528 may contact an opposite side of inner surface 590 from PCB 526.

In alternatives, imaging device 528 may not be coupled to an edge of PCB 526 and contacts for coupling to solder balls 582 may be longitudinally offset from one another on a surface of PCB 526. For example, a contact that is closer to inner surface 590 may be distal to a contact that is further from inner surface 590 (more radially inward). The contacts may also have such an arrangement when imaging device 528 is coupled to the edge of PCB 526.

An angle formed between longitudinal axis G of imaging device 528 and a longitudinal axis of PCB 526 may be from about 0 degrees to about 90 degrees, e.g., about 5 degrees to about 85 degrees, or about 10 degrees to about 50 degrees, or about 30 degrees. PCB 526 may be aligned approximately parallel to a longitudinal axis H of shaft 504. Accordingly, an angle formed between longitudinal axis G of imaging device 528 and longitudinal axis H of shaft 504 may be from about 0 degrees to 90 degrees, e.g., about 5 degrees to about 85 degrees, or about 10 degrees to about 50 degrees, or about 30 degrees with longitudinal axis H of shaft 504.

Distal portion 509 of shaft 504 may be filled with a material 562, similar to materials 162, 262, 362, 462, that cover or surround imaging device 528 and PCB 526. Material 562 may surround or cover portions of imaging device 528, except for a distally facing surface of a lens 545 of imaging device 528. In some examples, due to imaging device 528 being angled relative to longitudinal axis H of shaft 504, portions of imaging device 528 may protrude from a distal-most end 576 of shaft 504. Additionally, portions of material 562 may protrude from distal-most end 576 of shaft 504 to cover circumferential surfaces of imaging device 528 that extend beyond distal-most end 576 of shaft 504. Alternatively, imaging device 528 and PCB 526 may be disposed within shaft 504, such that no portion of imaging device 528 protrudes from distal-most end 576 of shaft 504.

Additionally, or optionally, imaging assembly 524 may include one or more light sources (e.g., light emitting diode(s)). For example, light source(s) may be disposed on either side of imaging device 528 to assist with visualization. During manufacturing of medical device 500, PCB 526 may be lengthened or shortened to provide proper alignment of imaging assembly 524 within distal portion 509 of shaft 504.

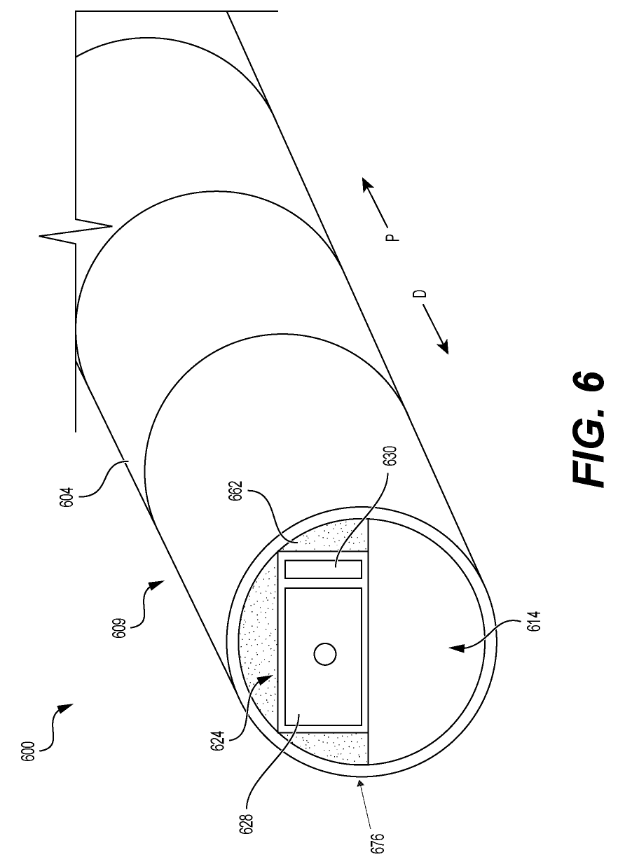

FIG. 6 illustrates a perspective view of a distal portion of an exemplary medical device 600 that includes a shaft 604 and an imaging assembly 624 disposed within a distal portion 609 of shaft 604. Shaft 604 may be used in combination with hub 102, e.g., in place of shaft 104 and cap 116.

Imaging assembly 624 may include an imaging device 628 and one or more light sources 630 (e.g., light emitting diode(s)). In this example, imaging device 628 and light source(s) 630 may be aligned parallel to a longitudinal axis of shaft 604. That is, imaging device 628 and light source(s) 630 may face a distal direction that is parallel to the longitudinal axis of shaft 604 (e.g., directly forward). Alternatively, imaging assembly 624 may be angled as described above. In some examples, the dimensions of imaging assembly 624 at a distal-most end 676 of shaft 604 may be approximately 1.1 x 0.75 mm, and an inner diameter of shaft 604 may be less than 2.0 mm.

A portion of distal portion 609 of shaft 604 may be filled with a material 662, similar to materials 162, 262, 362, 462, 562, that cover or surround imaging device 628 and light source(s) 630. Alternatively, imaging device 628 and light source(s) 630 may be separately mounted within distal portion 609 of shaft 604 using material 662. Although not illustrated, one or more wires or cables may extend proximally from imaging assembly 624 to, e.g., transmit signals, receive power, etc.

Shaft 604 may include a working channel 614 for receiving a guidewire or other medical instrument, delivering fluid (e.g., air or water), or applying suction, among other aspects. Working channel 614 may extend from a proximal end of shaft 604 to a distal opening at distal-most end 676 of shaft 604. For example, shaft 604 may be a multi-lumen extrusion with electronic components (imaging assembly 624, one or more wires or cables) contained in a lumen separate from working channel 614, which may help prevent fluid ingress into the electronic components. In an alternative, shaft 604 may include additional channels extending to distal-most end 676 of shaft 604. In another alternative, working channel 614 may be omitted, and inner portions of shaft 604 surrounding imaging assembly 624 may be filled with material 662.

As mentioned above, medical devices 100, 200, 300, 400, 500, 600 may be used in combination with other medical devices. For example, the shafts 104, 204, 304, 404, 504, 604 herein may be inserted into a shaft of another medical device. In these instances, medical devices 100, 200, 300, 400, 500, 600 may be reusable. For example, medical devices 100, 200, 300, 400, 500, 600 may be labeled as reusable, and may only need a simple “wipe-down cleaning” between uses.

Additionally, or optionally, shafts 104, 204, 304, 404, 504, 604 may have adequate lubricity to be insertable into or removable from another medical device or a body lumen. Additionally, or optionally, shafts 104, 204, 304, 404, 504, 604 may be flexible or semi-flexible, while maintaining integrity against unwanted rotation or compression during insertion into another medical device or a body lumen. Additionally, or optionally, respective shafts 104, 204, 304, 404, 504, 604 may include steering capabilities for deflecting or articulating respective distal ends of shafts 104, 204, 304, 404, 504, 604. For example, steering wires may extend through shafts 104, 204, 304, 404, 504, 604. Additionally, or optionally, shafts 104, 204, 304, 404, 504, 604 may be optically clear. Although medical devices 200, 300, 400, 500 are not illustrated with working channels, in alternatives, medical devices 200, 300, 400, 500 may include one or more channels extending through shafts 204, 304, 404, 504, for, e.g., receiving a guidewire or other medical instrument, delivering fluid (e.g., air or water), or applying suction, among other aspects. For example, the shape of PCB 326 described above may accommodate a working channel extending through shaft 304 of medical device 300.

An exemplary medical system 701 will now be described with reference to FIGS. 7A and 7B. Any of the medical devices described herein (i.e., medical devices 100, 200, 300, 400, 500, 600) may be used in combination with medical system 701. Although medical system 701 is described herein, it will be appreciated that the medical devices described above may be used with other systems or alone.

FIG. 7A illustrates a side view of medical system 701. Medical system 701 may include a medical device 703. Medical device 703 may include a handle 713 and a shaft 721. Handle 713 may include a gripping portion 715 configured for gripping by a user during a medical procedure. Handle 713 may be equipped with actuators 717 for controlling various functions of medical device 703, such as, e.g., deploying a needle, delivering vapor, delivering fluid (e.g., water or saline), etc. One or more fluidics tubes and cables 719 may extend from handle 713, e.g., to a controller, fluid source(s), and to couple medical device 703 to power or other electronic elements.

Shaft 721 may extend distally from handle 713 and may terminate in a distal tip 723. Shaft 721 may be an elongated structure designed for insertion into a subject's body (e.g., into a subject’s urethra) during medical procedures. Distal tip 723 may be configured for atraumatic insertion and navigation within bodily cavities or tissues. Shaft 721 may include a first lumen 725 and a second lumen 727. Handle 713 may include a port 735 in communication with second lumen 727. A needle 729 may extend through at least a portion of first lumen 725.

Needle 729 may be a hollow, elongated structure configured to deliver vapor to a tissue (e.g., to a tissue of a prostate). Needle 729 may terminate in a needle tip 731 at its distal end. A distal opening 733 may be present at distal tip 723 of shaft 721, through which needle tip 731 may protrude (e.g., radially outward relative to shaft 721). Distal opening 733 may be positioned on a side surface of shaft 721, allowing for lateral (radially outward) deployment of needle 729. For example, needle 729 may be retractable, allowing it to be withdrawn into first lumen 725 when not in use. In some aspects, one or more of actuators 717 of handle 713 may be configured for extending or retracting needle 729.

Any of the medical devices 100, 200, 300, 400, 500, 600 disclosed herein may be inserted into second lumen 727. A distal end of the respective medical device may be advanced to a distal end 780 of second lumen 727. Second lumen 727 may be in communication with opening 733 or another opening, such that tools or the like extended through medical device 703 may exit through opening 733 or another opening.

For example, as more clearly shown in FIG. 7B, distal opening 733 may define a distal opening for both first lumen 725 and second lumen 727. This configuration may allow for the deployment of instruments or delivery of fluids (e.g., cooling fluids) from first lumen 725, while also allowing for visualization of needle tip 731 by a respective imaging device 128, 228, 328, 428, 528, 628 of a respective imaging assembly 124, 224, 324, 424, 524, 624. The angle of the respective imaging assemblies 124, 224, 324, 424, 524, 624 may be such that the respective imaging devices 128, 228, 328, 428, 528, 628 may view needle 729 as it is extended and retracted.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A medical device comprising:
a hub;
a shaft extending distally from the hub to a distal end, the shaft comprising:
a working channel extending through the shaft and terminating at a distal opening proximal to a distal-most face of the shaft; and
a cap coupled to the distal end of the shaft, the cap comprising:
a first portion extending distally from the distal-most face of the shaft, wherein the first portion comprises an imaging assembly angled relative to a longitudinal axis of the cap, and wherein the imaging assembly comprises an imaging device and a light source mounted on a printed circuit board (PCB); and
a second portion received within the distal end of the shaft.

2. The medical device of claim 1, wherein the PCB comprises a distal-facing surface and a proximal-facing surface, and wherein each of the imaging device and the light source is mounted on the distal-facing surface of the PCB.

3. The medical device of claim 1, wherein the imaging assembly is disposed on an angled surface of the first portion.

4. The medical device of claim 1, wherein an angle of the imaging assembly relative the longitudinal axis of the cap is from about 10 degrees to about 50 degrees.

5. The medical device of claim 1, wherein the first portion of the cap comprises a distal-most surface angled relative to the longitudinal axis of the cap, wherein the imaging device extends to the distal-most surface, and wherein each of the PCB and the light source is proximal to the distal-most surface.

6. The medical device of claim 5, wherein the first portion of the cap comprises an encapsulate material completely covering each of a distal-facing surface of the PCB and the light source, and wherein the encapsulate material only partially covers the imaging device.

7. The medical device of claim 6, wherein the distal-most surface of the cap is a planar surface comprising a distally facing surface of a lens of the imaging device, and portions of the encapsulate material.

8. The medical device of claim 6, wherein the encapsulate material comprises an optically clear epoxy.

9. The medical device of claim 1, wherein the first portion comprises a body containing the imaging assembly, and wherein the second portion comprises a cylindrical body extending proximally from the body of the first portion.

10. The medical device of claim 9, wherein a cross-sectional shape of the body of the first portion is semi-annular or semi-circular.

11. The medical device of claim 1, wherein the working channel is a first channel, wherein the shaft further comprises a second channel terminating at a distal opening at the distal-most face of the shaft, and wherein the second portion of the shaft is received within the second channel.

12. The medical device of claim 11, wherein the cap comprises a channel extending proximally from the PCB, and wherein the channel of the cap is in communication with the second channel of the shaft.

13. The medical device of claim 12, wherein one or more wires extend proximally from the PCB, through the channel of the cap, through the second channel of the shaft, to the hub.

14. The medical device of claim 13, wherein the PCB is a first PCB, wherein the hub comprises a second PCB, and wherein the one or more wires extend proximally from the first PCB to the second PCB.

15. The medical device of claim 1, wherein the distal opening of the working channel is angled relative to the distal-most face of the shaft.

16. A cap for a medical device, the cap comprising:
a first portion comprising:
a body, wherein the body comprises an inner surface recessed from a distal-most end of the body, wherein the inner surface is angled relative to a longitudinal axis of the cap; and
an imaging assembly disposed on the inner surface of the body, wherein the imaging assembly comprises an imaging device and a light source mounted on a distal-facing surface of a printed circuit board (PCB), and wherein a proximal-facing surface of the PCB contacts the inner surface of the body;
a second portion comprising:
a cylindrical body extending proximally from the body of the first portion, wherein the cylindrical body is configured to be received within a distal end of a shaft of a medical device; and
an optically clear material within the body, wherein the optically clear material only covers portions of the imaging assembly.

17. The cap of claim 16, wherein a distal-most surface of the cap is a planar surface that comprises portions of the optically clear material and a distally facing surface of a lens of the imaging device that is free of the optically clear material.

18. A medical device comprising:
a shaft including a distal portion; and
a cap coupled to the distal portion of the shaft, the cap comprising:
a first portion extending distally from a distal-most end of the shaft, the first portion comprising:
a body, wherein the body comprises an inner wall recessed from a distal-most end of the body, wherein the inner wall comprises a distally facing surface that is angled relative to a longitudinal axis of the shaft; and
an imaging assembly disposed on the distally facing surface of the inner wall, wherein the imaging assembly comprises an imaging device and a light source mounted on a printed circuit board (PCB);
a second portion comprising a cylindrical body received within the distal portion of the shaft; and
an encapsulate material covering portions of the imaging assembly.

19. The medical device of claim 18, wherein the encapsulate material extends through an opening in the inner wall of the body of the first portion.

20. The medical device of claim 18, wherein the encapsulate material completely surrounds the cylindrical body of the second portion, and wherein the encapsulate material completely fills an interior of the cylindrical body.
